(19) United States
(12) Patent Application Publication
(10) Pub. No.: US 2023/0036899 A1
(43) Pub. Date: Feb. 2, 2023

(54) SURGICAL STAPLING DEVICE WITH EXTRUSION RESISTANT TOOL ASSEMBLY

(71) Applicant: Covidien LP, Mansfield, MA (US)

(72) Inventor: Jeffrey A. Miller, East Haven, CT (US)

(21) Appl. No.: 17/391,232

(22) Filed: Aug. 2, 2021

(51) Int. Cl. A61B 17/072

(52) U.S. Cl. CPC A61B 17/07292; A61B 2017/07285; A61B 2017/07264; A61B 2017/07271

(57) ABSTRACT

A surgical stapling device includes a tool assembly having an anvil assembly having a staple forming surface, a cartridge assembly having a tissue contact surface, and extrusion resistant structure. The extrusion resistant structure is positioned on only one of the staple forming surface of the anvil assembly or the tissue contact surface of the cartridge assembly to reduce the amount of tissue extrusion that occurs when the tool assembly is moved from an open position to a clamped position.

### FIELD

This technology is generally related to surgical stapling devices and, more particularly, to surgical stapling devices with extrusion resistant tool assemblies.

### BACKGROUND

Surgical stapling devices for endoscopic use are well known and commonly used to minimize patient trauma and reduce patient recovery times. Typically, endoscopic stapling devices include a tool assembly that has a first jaw that supports an anvil assembly and a second jaw that supports a cartridge assembly. The first and second jaws are secured together at their proximal ends by a pivot member such that the tool assembly can pivot between open and clamped positions. When tissue is positioned between the first and second jaws and the tool assembly is pivoted from the open position to the clamped position, the tissue can be extruded from a distal portion of the tool assembly.

During a lower anterior resection (“LAR”) procedure, a portion of the large intestine is removed to remove regions of cancer or diverticulitis from the large intestine or to treat inflammatory bowel disease. The diseased section of the bowel can be resected using a linear endoscopic surgical stapling device. Since access to the pelvic region to perform a LAR procedure can be difficult, it is preferential to resect the bowel using a single firing of the stapling device. Tissue extrusion from the tool assembly during clamping of tissue may prevent resection of the bowel in a single firing of the stapling device.

A continuing need exists in the art for a surgical stapling device that includes an extrusion resistant tool assembly.

### SUMMARY

This disclosure is directed to a surgical stapling device that includes a tool assembly having an anvil assembly with a staple forming surface, a cartridge assembly with a tissue contact surface, and extrusion resistant structure. The cartridge and anvil assemblies are coupled together at their proximal portions such that the tool assembly is pivotable from an open position to a clamped position. The extrusion resistant structure is positioned on only one of the staple forming surface of the anvil assembly or the tissue contact surface of the cartridge assembly to reduce the amount of tissue extrusion that occurs when the tool assembly is moved from the open position to the clamped position.

Aspects of this disclosure are directed to a tool assembly that includes an anvil assembly, a cartridge assembly, and extrusion resistant structure. The anvil assembly has a proximal portion and a staple forming surface. The staple forming surface defines a knife slot and staple forming pockets positioned on each side of the knife slot. The cartridge assembly includes a channel member and a staple cartridge. The proximal portion of the channel member is pivotably coupled to the proximal portion of the anvil assembly such that the tool assembly is movable from an open position to a clamped position. The channel member defines a cavity that receives the staple cartridge. The staple cartridge includes a body having a tissue contact surface that defines a knife slot and staple receiving slots positioned on each side of the knife slot. The extrusion resistant structure is positioned on only one of the staple forming surface of the anvil assembly or the tissue contact surface of the staple cartridge and is configured to grip tissue clamped between the anvil and cartridge assemblies to reduce tissue extrusion when the tool assembly is moved from the open position to the clamped position to clamp tissue.

In aspects of the disclosure, the extrusion resistant structure includes a mesh that is secured to the staple cartridge and extends across at least a portion of the tissue contact surface of the staple cartridge.

In some aspects of the disclosure, the mesh is secured to the staple cartridge with an adhesive.

In certain aspects of the disclosure, the mesh is formed from a self-fixating material.

In aspects of the disclosure, the mesh is formed from monofilament polyester and polybasic acid.

In some aspects of the disclosure, the extrusion resistant structure includes recessed treads that are positioned along the tissue contact surface of the staple cartridge.

In certain aspects of the disclosure, the recessed treads communicate with the knife slot of the body of the staple cartridge.

In aspects of the disclosure, the cartridge assembly defines a longitudinal axis, and the recessed treads are positioned diagonally along the tissue contact surface in relation to the longitudinal axis.

In some aspects of the disclosure, the extrusion resistant structure includes a barbed suture that is wrapped about the cartridge assembly.

In certain aspects of the disclosure, the extrusion resistant structure includes a roughened surface that is formed on the staple forming surface of the anvil assembly.

Other aspects of the disclosure are directed to a surgical stapling device including an elongate body and a tool assembly. The elongate body has a proximal portion and a distal portion. The tool assembly is supported on the distal portion of the elongate body and includes an anvil assembly, a cartridge assembly, and extrusion resistant structure. The anvil assembly has a proximal portion and a staple forming surface. The staple forming surface defines a knife slot and staple forming pockets positioned on each side of the knife slot. The cartridge assembly includes a channel member and a staple cartridge. The proximal portion of the channel member is pivotably coupled to the proximal portion of the anvil assembly such that the tool assembly is movable from an open position to a clamped position. The channel member defines a cavity that receives the staple cartridge. The staple cartridge includes a body having a tissue contact surface that defines a knife slot and staple receiving slots positioned on each side of the knife slot. The extrusion resistant structure is positioned on only one of the staple forming surface of the anvil assembly or the tissue contact surface of the staple cartridge and is configured to grip tissue clamped between the anvil and cartridge assemblies to reduce tissue extrusion when the tool assembly is moved from the open position to the clamped position to clamp tissue.

In aspects of the disclosure, the surgical stapling device includes a handle assembly, and the proximal portion of the elongate body is coupled to the handle assembly.

Still other aspects of the disclosure are directed to a tool assembly including an anvil assembly, a cartridge assembly, and extrusion resistant structure. The anvil assembly has a proximal portion and a staple forming surface. The cartridge assembly includes a staple cartridge and is pivotably coupled to the proximal portion of the anvil assembly such that the tool assembly is movable from an open position to a clamped position. The staple cartridge includes a body having a tissue contact surface. The extrusion resistant structure is positioned on only one of the staple forming surface of the anvil assembly or the tissue contact surface of the staple cartridge and is configured to grip tissue clamped between the anvil and cartridge assemblies to reduce tissue extrusion when the tool assembly is moved from the open position to the clamped position to clamp tissue.

Other features of the disclosure will be appreciated from the following description.

### BRIEF DESCRIPTION OF DRAWINGS

Various aspects of the disclosure are described herein below with reference to the drawings, wherein:

FIG. 1 is a side perspective view of a surgical stapling device according to aspects of the disclosure;

FIG. 2 is a side perspective view of a tool assembly of the surgical stapling device shown in FIG. 1 illustrating a tissue contact surface of a cartridge assembly of the tool assembly with the tool assembly in an open position;

FIG. 2A is a side perspective view of the tool assembly of the surgical stapling device shown in FIG. 1 illustrating a staple forming surface of the anvil assembly with the tool assembly in an open position;

FIG. 3 is a side view of the tool assembly shown in FIG. 2 with an extrusion resistant structure separated from a cartridge assembly of the tool assembly;

FIG. 4 is a side view of the tool assembly shown in FIG. 2 with the tool assembly in the open position;

FIG. 5 is a side view of the tool assembly shown in FIG. 2 with the tool assembly in the clamped position;

FIG. 6 is a perspective view of a distal portion of a cartridge assembly of an alternate version of the tool assembly of the surgical stapling device shown in FIG. 1;

FIG. 7 is a perspective view of a distal portion of a cartridge assembly of another alternate version of the tool assembly of the surgical stapling device shown in FIG. 1; and

FIG. 8 is a side perspective view of a distal portion of another alternate version of the tool assembly of the surgical stapling device shown in FIG. 1.

### DETAILED DESCRIPTION

The disclosed surgical stapling device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary manner, while the term “distal” is used generally to refer to that portion of the device that is farther from the clinician during use of the device in its customary manner. In addition, the term “endoscopic” is used generally used to refer to endoscopic, laparoscopic, arthroscopic, and/or any other procedure conducted through a small diameter incision or cannula. Further, the term “clinician” is used generally to refer to medical personnel including doctors, nurses, and support personnel. Moreover, directional terms such as front, rear, upper, lower, top, bottom, distal, proximal, and similar terms are used to assist in understanding the description and are not intended to limit the disclosure.

This application is directed to a surgical stapling device that includes a tool assembly having an anvil assembly with a staple forming surface, a cartridge assembly with a tissue contact surface, and extrusion resistant structure. The anvil and cartridge assemblies are coupled together at their proximal portions such that the tool assembly is pivotable from an open position to a clamped position. The extrusion resistant structure is positioned on only one of the staple forming surface of the anvil assembly or the tissue contact surface of the cartridge assembly to reduce the amount of tissue extrusion that occurs when the tool assembly is moved from the open position to the clamped position.

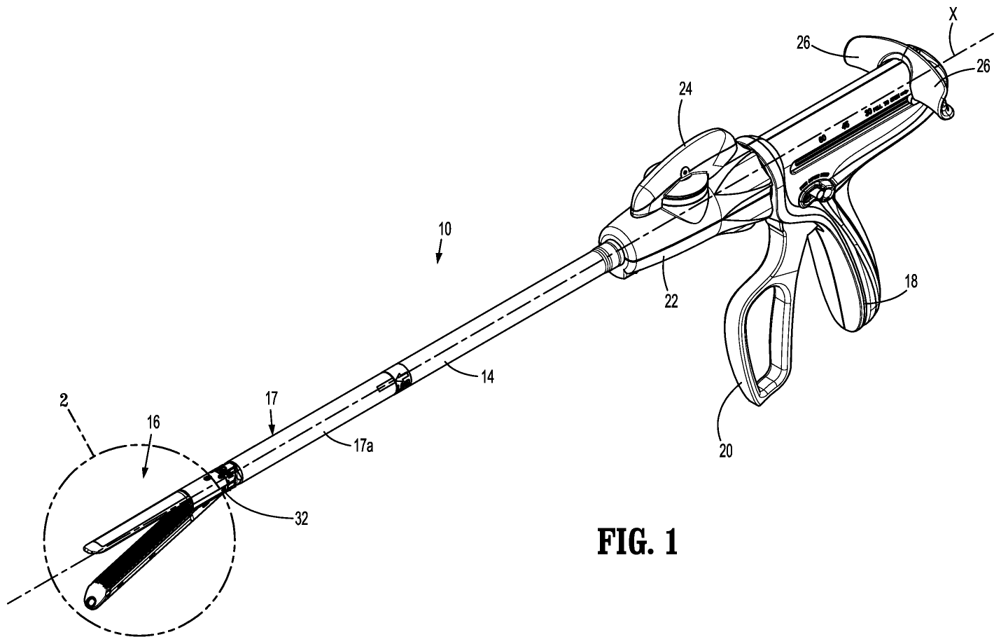

FIG. 1 illustrates exemplary aspects of the disclosed surgical stapling device shown generally as stapling device 10. The stapling device 10 includes a handle assembly 12, an elongate body 14 defining a longitudinal axis “X” (FIG. 1), and a tool assembly 16. The handle assembly 12 includes a stationary handle 18 and a trigger 20 that is movable in relation to the stationary handle 18 to actuate the tool assembly 16. In aspects of the disclosure, the stapling device 10 includes a rotation knob 22 and an articulation lever 24. The rotation knob 22 is coupled to the handle assembly 12 and supports the elongate body 14 to facilitate rotation of the elongate body 14 and the tool assembly 16 in relation to the handle assembly 12 about the longitudinal axis “X”. The articulation lever 24 is coupled to the tool assembly 16 to articulate the tool assembly 16 from a position aligned with the longitudinal axis “X” to positions defining acute angles with the longitudinal axis “X”. In some aspects of the disclosure, the handle assembly 12 includes retraction knobs 26 for retracting a drive assembly (not shown) of the stapling device 10. For a more detailed description of exemplary aspects of the handle assembly 12 of the stapling device 10, see, e.g., U.S. Pat. No. 6,241,139 (“the '139 Patent”). Although the handle assembly 12 is illustrated as a manually operated handle assembly, it is envisioned that the handle assembly 12 could also be a powered handle assembly such as described in U.S. Pat. No. 9,055,943.

In aspects of the disclosure, the tool assembly 16 forms part of a reload assembly 17 that includes the tool assembly 16 and a body portion 17a. The body portion 17a has a proximal end that is adapted to be releasably coupled to the elongate body 14, and a distal end that supports the tool assembly 16. Alternately, the elongate body 14 and the body portion 17a can be formed as an integral unit.

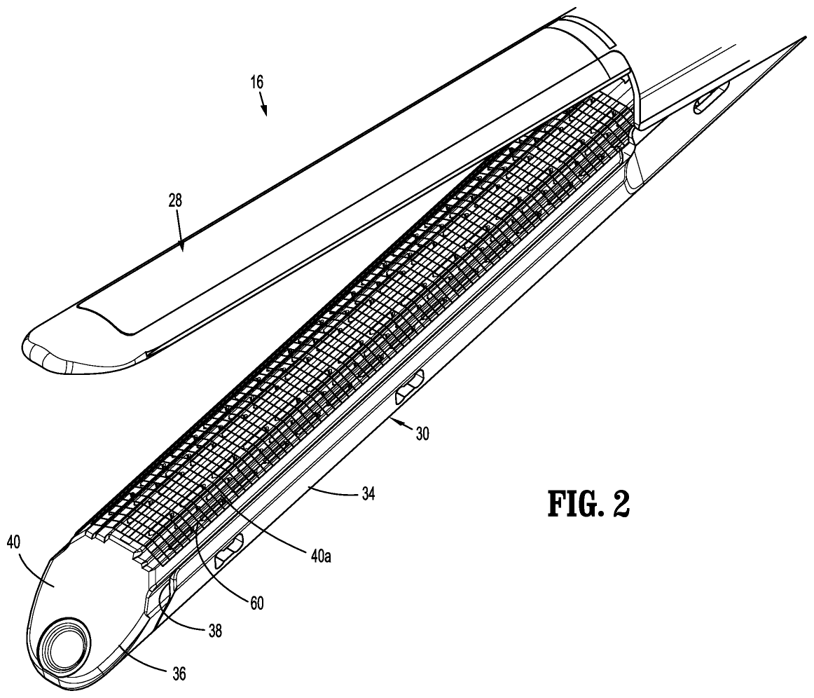

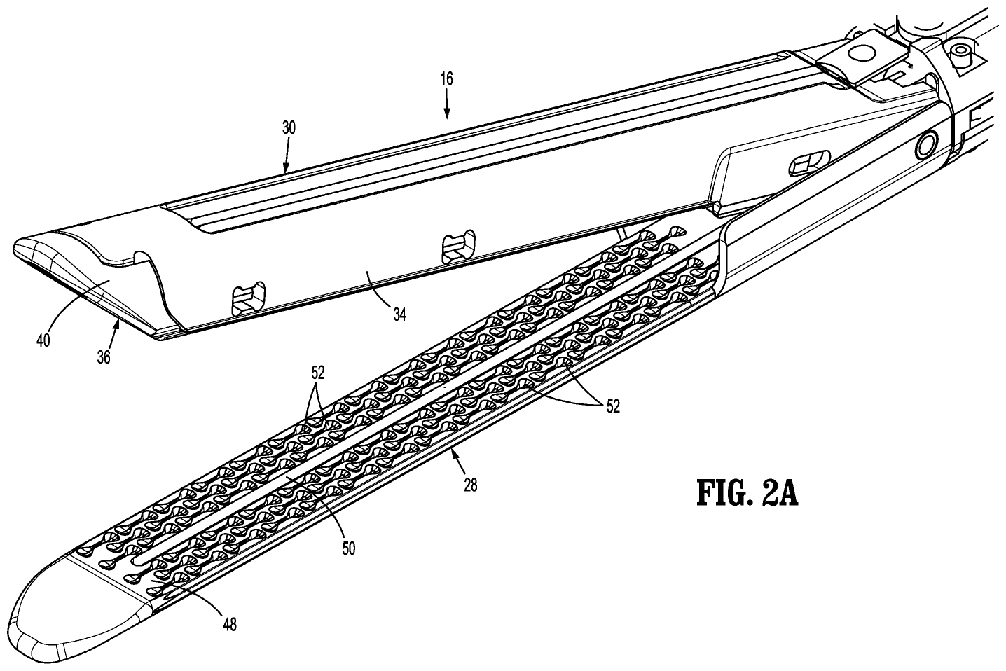

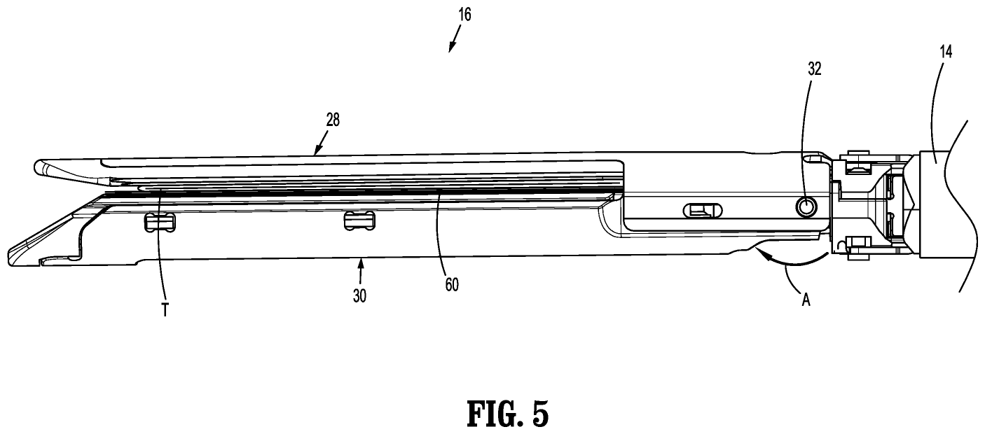

FIGS. 2 and 3 illustrate the tool assembly 16 of the stapling device 10 (FIG. 1). The tool assembly 16 includes an anvil assembly 28 and a cartridge assembly 30 that are movable in relation to each other between an open position (FIG. 1) and a clamped position (FIG. 5). In aspects of the disclosure, proximal portions of the anvil and cartridge assemblies 28, 30 are coupled together with pivot members 32 (FIG. 1) (only one is shown) such that the cartridge assembly 30 can pivot in relation to the anvil assembly 28 between an open position (FIG. 1) and a clamped position (FIG. 5). Although the cartridge assembly 30 is illustrated as pivoting towards a stationary anvil assembly 28, it is envisioned that the anvil assembly 28 could be adapted to pivot towards a stationary cartridge assembly 30.

The cartridge assembly 30 includes a channel member 34 and a staple cartridge 36. The channel member 34 defines a cavity 38 (FIG. 2) that receives the staple cartridge 36. In aspects of the disclosure, the staple cartridge 36 can be replaced after each firing to facilitate reuse of the stapling device 10 (FIG. 1). Alternately, the staple cartridge 36 can be fixedly retained within the cavity 38 of the channel member 34. The staple cartridge 36 includes a body 40 that has a tissue contact surface 40a and defines staple receiving slots 42 and a knife slot 44. In aspects of the disclosure, the staple receiving slots 42 are positioned in rows, e.g., three rows, on each side of the knife slot 44. Each of the staple receiving slots 42 receives a staple (not shown) and a pusher (not shown). The staple cartridge 36 also includes an actuation sled (not shown) that is movable through the body 40 of the staple cartridge 36 to eject the staples from the staple receiving slots 42.

The anvil assembly 28 includes a staple forming surface 48 (FIG. 2A) that defines a central knife slot 50 and a plurality of staple forming pockets 52 (FIG. 9) positioned on each side of the central knife slot 50. In aspects of the disclosure, the staple forming pockets 52 are aligned in rows, e.g., three rows, on each side of the central knife slot 50 and are aligned with the staple receiving slots 42 when the tool assembly 16 is in the clamped position.

In aspects of the disclosure, the tool assembly 16 supports extrusion resistant structure that is positioned on the tissue contact surface 40a of the staple cartridge 36 or the staple forming surface 48 of the anvil assembly 28. In aspects of the disclosure, the extrusion resistant structure forms a textured surface on the tissue contact surface 40a of the staple cartridge 36 or the staple forming surface 48 of the anvil assembly 28 to increase friction on the surface of the anvil assembly 28 or the staple cartridge 36 of the cartridge assembly 30. Increasing the friction on only one of the tissue contact surface 40a of the staple cartridge 36 or the staple forming surface 48 of the anvil assembly 28 allows the anvil assembly 28 or cartridge assembly 30 to slide in relation to tissue during movement of the tool assembly 16 from the open position to the clamped position while reducing the amount of tissue extrusion from the distal portion of the tool assembly 16.

FIGS. 2-5 illustrate the tool assembly 16 in which the extrusion resistant structure includes a mesh 60 that is secured or adhered to the staple cartridge 36 and extends at least partially across the tissue contact surface 40a of the staple cartridge 36, e.g., across the entire tissue contact surface 40a of the staple cartridge 36. In some aspects of the disclosure, the mesh 60 is secured to the staple cartridge 36 using an adhesive or tacky substance that allows the mesh 60 to be released from the staple cartridge 36 after the stapling device 10 has been fired. In certain aspects of the disclosure, the adhesive or tacky substance can be incorporated into or onto the mesh 60. Alternately, the mesh 60 can be secured to anvil assembly 28 or staple cartridge 36 of the tool assembly 16 using a mechanical fastener. The mesh 60 can be formed of any biocompatible material that increases friction of the tissue contact surface 40a of the staple cartridge 36. In some aspects of the disclosure, the mesh 60 is formed from a polyester material. In certain aspects of the disclosure, the mesh 60 is formed from a self-fixating material such as PROGRIP™ Laparoscopic Self-Fixating Mesh which is formed of monofilament polyester and polylactic acid.

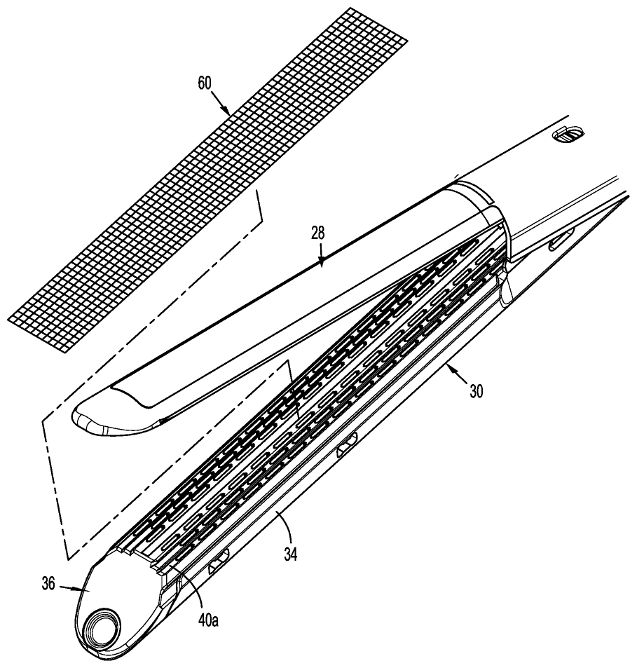

When tissue “T” is placed between the tissue contact surface 40a of the staple cartridge 36 and the staple forming surface 48 of the anvil assembly 28 (FIG. 4), and the tool assembly 16 is moved to the clamped position, the mesh 60 grips the tissue “T” to reduce the amount of tissue “T” extrusion from the distal portion of the tool assembly 16. As the cartridge assembly 30 pivots about the pivot members 32 in the direction of arrow “A” in FIG. 5, the mesh 60 engages the tissue “T” to minimize slippage of the tissue “T” along the tissue contact surface 40a (FIG. 2) of the staple cartridge 36. As the tissue “T” is compressed against the staple forming surface 48 of the anvil assembly 28, the tissue “T” can slide along the staple forming surface 48 of the anvil assembly 28 as the tool assembly 16 moves to the clamped position.

Once the tool assembly 16 is properly clamped about the tissue “T”, the stapling device 10 can be fired to eject staples (not shown) from the staple cartridge 36. The staples pass through the mesh 60 to secure the mesh 60 to the tissue “T”. The mesh 60 disengages from the staple cartridge 36 when tool assembly 16 of the stapling device 10 (FIG. 1) is opened and the stapling device 10 is removed from a patient. For a detailed description of firing of an exemplary stapling device, see the '139 Patent.

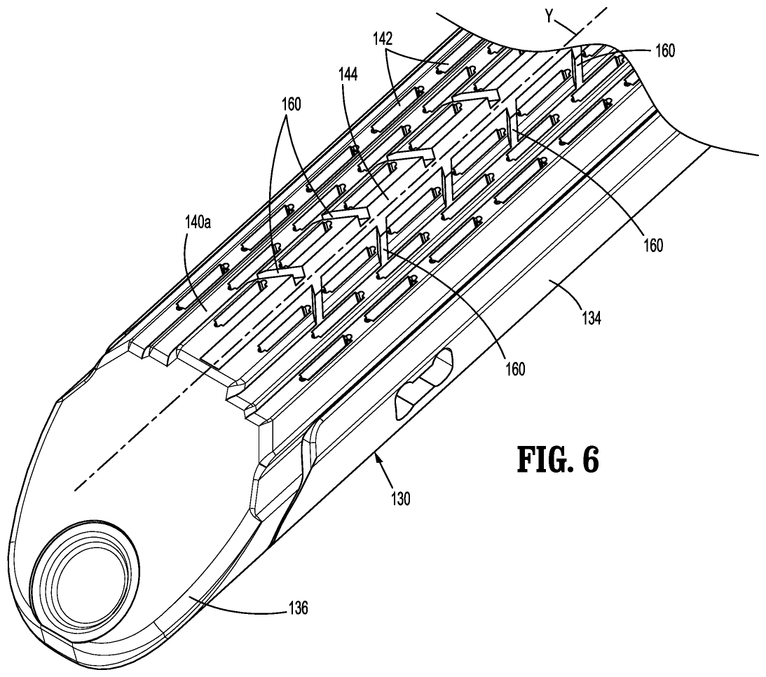

FIG. 6 illustrates an alternate version of the cartridge assembly of the stapling device 10 shown generally as cartridge assembly 130. The cartridge assembly 130 includes a channel member 134 and a staple cartridge 136. The channel member 134 defines a cavity 138 that receives the staple cartridge 136. In aspects of the disclosure, the staple cartridge 136 can be replaced after each firing to facilitate reuse of the stapling device 10 (FIG. 1). The staple cartridge 136 includes a body 140 that has a tissue contact surface 140a and defines staple receiving slots 142 and a knife slot 144. In aspects of the disclosure, the staple receiving slots 142 are positioned in rows, e.g., three rows, on each side of the knife slot 144. Each of the staple receiving slots 142 receives a staple (not shown) and a pusher (not shown). The staple cartridge 136 also includes an actuation sled (not shown) that is movable through the body 140 of the staple cartridge 136 to eject the staples from the staple receiving slots 142.

In contrast to the staple cartridge 36 (FIG. 2) of the cartridge assembly 30 described above, the staple cartridge 136 of the cartridge assembly 130 is formed with the extrusion resistant structure which includes recessed treads 160 formed along the tissue contact surface 140a of the staple cartridge 136. In aspects of the disclosure, the recessed treads 160 are formed along a substantial length of the staple cartridge 136 and communicate with the knife slot 144. In some aspects of the disclosure, the recessed treads 160 are positioned inwardly of the outer rows of staple receiving slots 142 and extend diagonally along the tissue contact surface 140a in relation to a longitudinal axis “Y” of the staple cartridge 136. In aspects of the disclosure, the recessed treads are angled towards the knife slot 144 in a proximal direction. The recessed treads 160 function to grip tissue positioned on the tissue contact surface 140a of the staple cartridge 136 in the same manner as the mesh 60 and will not be described in further detail herein.

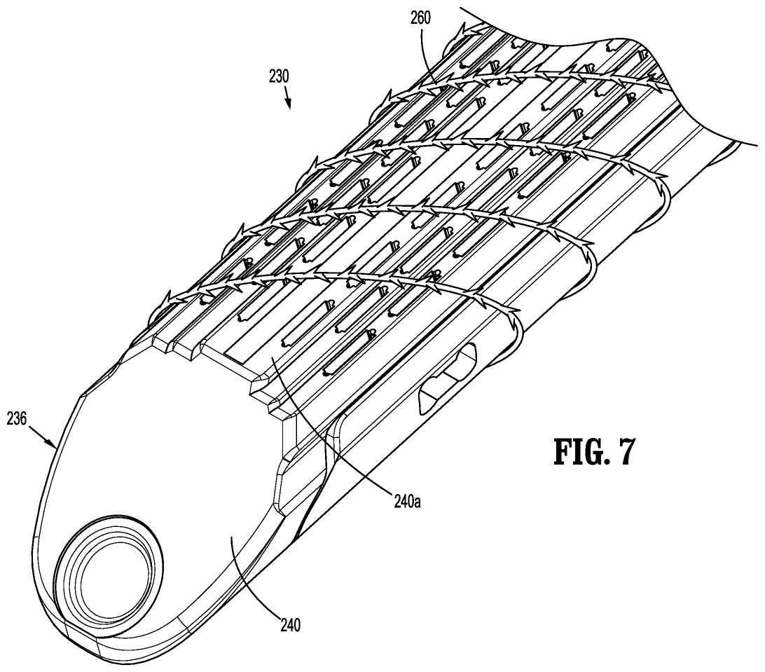

FIG. 7 illustrates an alternate version of the cartridge assembly of the stapling device 10 shown generally as cartridge assembly 230. In contrast to the staple cartridges 36 (FIG. 2) and 136 (FIG. 6) described above, the cartridge assembly 230 includes extrusion resistant structure in the form of a barbed suture 260 that is wrapped or wound about the cartridge assembly 230 and extends across the tissue contact surface 240a of the body 240 of the staple cartridge 236 along at least a portion of the length of the staple cartridge 236. The barbed suture 260 functions to grip tissue positioned on the tissue contact surface 240a of the staple cartridge 236 in a manner like the mesh 60 (FIG. 2) and the recessed treads 160 (FIG. 6) and will not be described in further detail herein. After the stapling device 10 is fired, the clinician can cut and remove portions of the suture 260 from the sutured tissue.

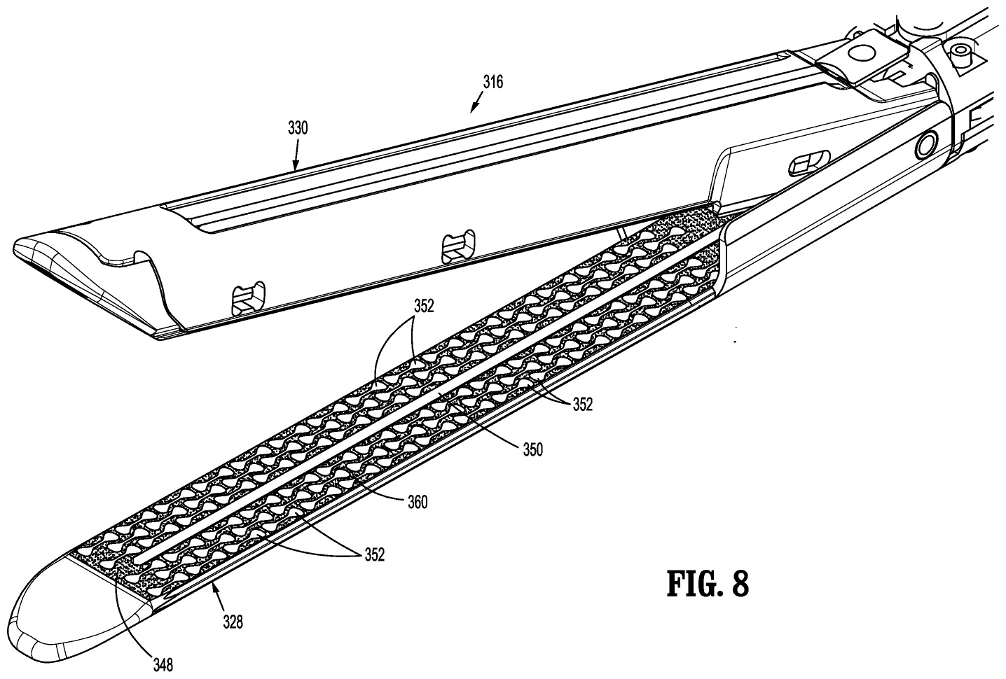

FIG. 8 illustrates an alternate version of the tool assembly 316 of the stapling device 10 (FIG. 1) shown generally as tool assembly 316. The tool assembly 316 includes an anvil assembly 328 and a cartridge assembly 330. The cartridge assembly 330 which is substantially like the cartridge assembly 30 (FIG. 2) without the mesh 60 will not be described in further detail herein. The anvil assembly 328 includes a staple forming surface 348 that defines a central knife slot 350 and a plurality of staple forming pockets 352 (FIG. 9) positioned on each side of the central knife slot 350. In aspects of the disclosure, the staple forming pockets 352 are aligned in rows, e.g., three rows, on each side of the central knife slot 350 and are aligned with the staple receiving slots (not shown) of the cartridge assembly 330 when the tool assembly 16 is in the clamped position.

The anvil assembly 328 includes extrusion resistant structure in the form of a roughened surface 360 that is positioned on the staple forming surface 348. In aspects of the disclosure, the roughened surface 360 of the anvil assembly 328 can be formed by abrading, sandblasting, laser etching, or other chemical or mechanical method to increase the surface roughness of the staple forming surface 348 of the anvil assembly 328. The roughened surface 360 of the anvil assembly functions to grip tissue positioned on the staple forming surface 348 of the anvil assembly 328 in a manner like the mesh 60 (FIG. 2), the recessed treads 160 (FIG. 6), and the barbed suture 260 (FIG. 7) and will not be described in further detail herein.

Although not described in detail herein, it is envisioned that the tissue contact surface of the staple cartridge can be formed with roughened surface instead of the staple forming surface of the anvil assembly to reduce the amount of tissue extrusion during movement of the tool assembly from its open position to its clamped position.

It is envisioned that the tool assembly described herein could be incorporated onto a robotic device and need not include a handle assembly. It is also envisioned that the tool assembly need not be a stapling device but rather could be any surgical device that tends to extrude tissue when the device is moved from an open position to a clamped position.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A tool assembly comprising:
an anvil assembly having a proximal portion and a staple forming surface, the staple forming surface defining a knife slot and staple forming pockets on each side of the knife slot;
a cartridge assembly including a channel member and a staple cartridge, the proximal portion of the channel member pivotably coupled to the proximal portion of the anvil assembly such that the tool assembly is movable from an open position to a clamped position, the channel member defining a cavity that receives the staple cartridge, the staple cartridge including a body having a tissue contact surface that defines a knife slot and staple receiving slots positioned on each side of the knife slot of the staple cartridge; and
extrusion resistant structure positioned on only one of the staple forming surface of the anvil assembly or the tissue contact surface of the staple cartridge, the extrusion resistant structure configured to grip tissue clamped between the anvil and cartridge assemblies to reduce tissue extrusion when the tool assembly is moved from the open position to the clamped position to clamp tissue.

2. The tool assembly of claim 1, wherein the extrusion resistant structure includes a mesh secured to the staple cartridge, the mesh extending across at least a portion of the tissue contact surface of the staple cartridge.

3. The tool assembly of claim 2, wherein the mesh is secured to the staple cartridge with an adhesive.

4. The tool assembly of claim 2, wherein the mesh is formed from a self-fixating material.

5. The tool assembly of claim 4, wherein the mesh is formed from monofilament polyester and polybasic acid.

6. The tool assembly of claim 1, wherein the extrusion resistant structure includes recessed treads positioned along the tissue contact surface of the staple cartridge.

7. The tool assembly of claim 6, wherein the recessed treads communicate with the knife slot of the body of the staple cartridge.

8. The tool assembly of claim 7, wherein the cartridge assembly defines a longitudinal axis, and the recessed treads are positioned diagonally along the tissue contact surface in relation to the longitudinal axis.

9. The tool assembly of claim 1, wherein the extrusion resistant structure includes a barbed suture that is wrapped about the cartridge assembly.

10. The tool assembly of claim 1, wherein the extrusion resistant structure includes a roughened surface formed on the staple forming surface of the anvil assembly.

11. A surgical stapling device comprising:
an elongate body having a proximal portion and a distal portion; and
a tool assembly supported on the distal portion of the elongate body, the tool assembly including:
an anvil assembly having a proximal portion and a staple forming surface, the staple forming surface defining a knife slot and staple forming pockets on each side of the knife slot;
a cartridge assembly including a channel member and a staple cartridge, the proximal portion of the channel member pivotably coupled to the proximal portion of the anvil assembly such that the tool assembly is movable from an open position to a clamped position, the channel member defining a cavity that receives the staple cartridge, the staple cartridge including a body having a tissue contact surface that defines a knife slot and staple receiving slots positioned on each side of the knife slot of the staple cartridge; and
extrusion resistant structure positioned on only one of the staple forming surface of the anvil assembly or the tissue contact surface of the staple cartridge, the extrusion resistant structure configured to grip tissue clamped between the anvil and cartridge assemblies to reduce tissue extrusion when the tool assembly is moved from the open position to the clamped position to clamp tissue.

12. The surgical stapling device of claim 11, wherein the extrusion resistant structure includes a mesh secured to the staple cartridge, the mesh extending across at least a portion of the tissue contact surface of the staple cartridge.

13. The surgical stapling device of claim 12, wherein the mesh is formed from a self-fixating material.

14. The surgical stapling device of claim 11, wherein the extrusion resistant structure includes recessed treads positioned along the tissue contact surface of the staple cartridge.

15. The surgical stapling device of claim 14, wherein the recessed treads communicate with the knife slot of the body of the staple cartridge.

16. The surgical stapling device of claim 15, wherein the cartridge assembly defines a longitudinal axis, and the recessed treads are positioned diagonally along the tissue contact surface in relation to the longitudinal axis.

17. The surgical stapling device of claim 11, wherein the extrusion resistant structure includes a barbed suture that is wrapped about the cartridge assembly.

18. The surgical stapling device of claim 11, wherein the extrusion resistant structure includes a roughened surface formed on the staple forming surface of the anvil assembly.

19. The surgical stapling device of claim 11, further including a handle assembly, the proximal portion of the elongate body coupled to the handle assembly.

20. A tool assembly comprising:
an anvil assembly having a proximal portion and a staple forming surface;
a cartridge assembly including a staple cartridge, the cartridge assembly pivotably coupled to the proximal portion of the anvil assembly such that the tool assembly is movable from an open position to a clamped position, the staple cartridge including a body having a tissue contact surface; and
extrusion resistant structure positioned on only one of the staple forming surface of the anvil assembly or the tissue contact surface of the staple cartridge, the extrusion resistant structure configured to grip tissue clamped between the anvil and cartridge assemblies to reduce tissue extrusion when the tool assembly is moved from the open position to the clamped position to clamp tissue.
